(19) United States
(12) Patent Application Publication
(10) Pub. No.: US 2025/0017726 A1
(43) Pub. Date: Jan. 16, 2025

(54) ADJUSTABLE LEAFLET ASSEMBLIES FOR EXPANDABLE PROSTHETIC HEART VALVES

(71) Applicant: EDWARDS LIFESCIENCES CORPORATION, Irvine, CA (US)

(72) Inventors: Hanoch Cohen-Tzemach, Holon (IL); Eran Goldberg, Nesher (IL)

(21) Appl. No.: 18/896,469

(22) Filed: Sep. 25, 2024

Related U.S. Application Data
(63) Continuation of application No. PCT/US2023/018265, filed on Apr. 12, 2023.
(60) Provisional application No. 63/362,956, filed on Apr. 13, 2022.

(51) Int. Cl. A61F 2/24

(52) U.S. Cl. CPC A61F 2/2418; A61F 2/243; A61F 2220/0075; A61F 2230/0013; A61F 2230/0054; A61F 2230/0069; A61F 2250/0004

(57) ABSTRACT

A prosthetic heart valve including a plurality of leaflets attached to a radially expandable frame is disclosed. The plurality of leaflets is disposed within the frame and configured to regulate a flow of blood through the frame in one direction. Each leaflet is folded along an axially extending fold to form an inner layer and an outer layer. The inner layer of each leaflet is configured to coapt with the inner layers of the other leaflets. A length of the inner layer in a circumferential direction progressively increases upon radial expansion of the frame.

### CROSS-REFERENCE TO RELATED APPLICATIONS

This application is a continuation of PCT patent application no. PCT/US2023/018265 filed on Apr. 12, 2023, which claims the benefit of U.S. Provisional Application No. 63/362,956, filed Apr. 13, 2022, each of which is incorporated herein in its entirety by this specific reference.

### FIELD

The present disclosure relates to prosthetic heart valves and systems and methods for prosthetic heart valves having adjustable leaflets that can be deployed to a range of working diameters.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

The annulus sizes of native valves can vary depending on the patient. To accommodate a range of annulus sizes, prosthetic heart valves can be manufactured in different, distinct sizes (e.g., different diameters, etc.). Typically, a prosthetic valve may be expanded to one only functional size when deployed at the implantation site in the heart. As such, a plurality of prosthetic valves are needed for treating patients having different annulus sizes. Thus, a prosthetic valve that can be expanded to any working diameter within a range of working diameters for treating patients with different annulus sizes would be desirable.

### SUMMARY

Described herein are prosthetic heart valves, delivery apparatus, and methods for assembling and implanting prosthetic heart valves. The disclosed prosthetic heart valves, delivery apparatus, and methods can, for example, provide versatility in that an expandable prosthetic heart valve has leaflets that are folded in a self-adjustable manner to permit the prosthetic valve to be expanded to a desired diameter within a range of working diameters. As such, the devices and methods disclosed herein can, among other things, overcome one or more of the deficiencies of typical prosthetic heart valves.

A prosthetic heart valve can comprise a frame and a valvular structure comprising a plurality of leaflets coupled to the frame. In addition to these components, a prosthetic heart valve can further comprise one or more of the components disclosed herein.

In some examples, a prosthetic heart valve can comprise a length of a coaptation portion of a leaflet in a circumferential direction progressively increases upon radial expansion of the frame.

In some examples, a prosthetic heart valve can comprise a leaflet having a longitudinal or axially-extending fold (e.g., a fold extending from an inflow edge to an outflow end of the leaflet) that defines an inner layer and an outer layer of the leaflet.

In some examples, a prosthetic heart valve comprises one or more of the components recited in Examples 1-60 below.

In one representative example, a prosthetic heart valve comprises a radially expandable frame comprising a plurality of interconnected struts; and a plurality of leaflets disposed within the frame and configured to regulate a flow of blood through the frame in one direction, wherein each leaflet is folded along an axially extending fold to form an inner layer and an outer layer, wherein the inner layer of each leaflet is configured to coapt with the inner layers of the other leaflets, and wherein a length of the inner layer in a circumferential direction progressively increases upon radial expansion of the frame.

In another representative example, a prosthetic heart valve comprises a frame comprising a plurality of interconnected struts, wherein the frame is radially expandable between at least a first diameter and a larger, second diameter; and a plurality of leaflets disposed within the frame and configured to regulate a flow of blood through the frame in one direction when the frame is radially expanded to the first diameter and when the frame is radially expanded to the second diameter, wherein each leaflet is folded along a longitudinal fold to form an inner layer and an outer layer, wherein each longitudinal fold defines a commissure for a pair of adjacent inner layers of two adjacent leaflets, wherein the inner layer of each leaflet defines a coaptation edge configured to move inwardly and outwardly in a radial direction relative to the outer layer at the commissure.

In another representative example, a prosthetic heart valve comprises a radially expandable frame comprising a plurality of interconnected struts; and a plurality of self-adjustable leaflets disposed within the frame, each leaflet comprising a longitudinal fold defining an outer layer and an inner layer, wherein an inner layer of a first leaflet includes a non-coaptation portion and a coaptation portion, wherein the non-coaptation portion of the first leaflet is disposed radially outward of an outer layer of a second leaflet, wherein each coaptation portion is configured to move inwardly and outwardly in a radial direction relative to the outer layer at the longitudinal fold.

In another representative example, a prosthetic heart valve comprises a frame including a plurality of leaflet attachment members; and a leaflet construct disposed within the frame and attached to the frame at the plurality of leaflet attachment members, the leaflet construct defining a leaflet between each of the plurality of leaflet attachment members, wherein each leaflet includes a longitudinal fold defining an inner layer and an outer layer, wherein each fold is circumferentially offset from the plurality of leaflet attachment members, wherein the inner layers of the leaflets are configured to move inward and outward in a radial direction relative to the frame to regulate the flow of blood through the frame.

In another representative example, a prosthetic heart valve comprises a radially expandable frame; and a leaflet construct disposed within the frame and including a plurality of leaflets, each leaflet including a longitudinal fold defining an inner layer and an outer layer, each inner layer having a non-coaptation segment and a coaptation segment, wherein a first outer layer is connected to a coaptation segment of a first inner layer and a non-coaptation segment of a second inner layer, wherein the first outer layer is disposed radially outward of the coaptation segment of the first inner layer, wherein the first outer layer is disposed radially inward of the non-coaptation segment of the second inner layer, wherein a length of the inner layers in a circumferential direction progressively increases upon radial expansion of the leaflet assembly, wherein a length of the outer layers in a circumferential direction progressively decreases upon radial expansion of the leaflet assembly.

In another representative example, a leaflet construct for a prosthetic heart valve comprises three inner layers, each inner layer having a non-coaptation segment and a coaptation segment; three outer layers coupled to the three inner layers, wherein a first outer layer of the three outer layers is connected to a coaptation segment of a first inner layer and a non-coaptation segment of a second inner layer, wherein the first outer layer is disposed radially outward of the coaptation segment of the first inner layer, wherein a second outer layer of the three outer layers is connected to a coaptation segment of the second inner layer and a non-coaptation segment of a third inner layer, wherein the second outer layer is disposed radially inward of the non-coaptation segment of the third inner layer; at least one longitudinal fold positioned between each inner layer and a corresponding outer layer; wherein a length of the inner layers in a circumferential direction progressively increases upon radial expansion of the leaflet construct; and wherein a length of the outer layers in a circumferential direction progressively decreases upon radial expansion of the leaflet construct.

In another representative example, a delivery assembly comprises a delivery apparatus including an expansion mechanism; and a prosthetic heart valve coupled to the delivery apparatus, the prosthetic heart valve comprises a radially expandable frame; and a plurality of leaflets disposed within the frame and configured to regulate a flow of blood through the frame in one direction, wherein each leaflet is folded along a longitudinal fold to form an inner layer and an outer layer, wherein the inner layer of each leaflet is configured to coapt with the inner layers of the other leaflets, and wherein a length of the inner layer in a circumferential direction progressively increases upon radial expansion of the frame by the expansion mechanism.

In another representative example, a method of implantation, comprises inserting a delivery device into a vessel of a patient, the delivery device including a radially expandable prosthetic heart valve; and expanding the prosthetic heart valve within or adjacent a native valve of a heart of the patient to a first diameter of a range of diameters of the prosthetic heart valve, the prosthetic heart valve including leaflets having folds defining inner layers of leaflets and outer layers of leaflets which slide relative to each other upon expansion of the prosthetic heart valve.

In another representative example, a method of delivering an implant within a patient's body, the implant comprising a prosthetic heart valve of any of the examples herein, the method comprises positioning the prosthetic heart valve at an implantation site within the patient's body; and expanding the prosthetic heart valve to a working diameter within a range of working diameters of the prosthetic heart valve.

The above method(s) can be performed on a living animal or on a simulation, such as on a cadaver, cadaver heart, anthropomorphic ghost, simulator (e.g., with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side elevation view of a prosthetic heart valve, according to one example.

FIG. 2 is a side view of an example of a delivery apparatus configured to deliver and implant a radially expandable prosthetic heart valve at an implantation site.

FIG. 3 is a plan view of an outflow end of a prosthetic heart valve with leaflets in an open configuration, according to another example.

FIG. 4 is a plan view of the outflow end of the prosthetic heart valve of FIG. 3 with the leaflets in a closed configuration.

FIG. 5A is a plan view of the outflow end of the prosthetic heart valve of FIG. 3 expanded to a first working diameter.

FIG. 5B is a plan view of the outflow end of the prosthetic heart valve of FIG. 3 expanded to a second working diameter that is larger than the first working diameter.

FIG. 5C is a plan view of the outflow end of the prosthetic heart valve of FIG. 3 fully expanded to a third, maximum working diameter.

FIG. 6 is a schematic, partial side view of the prosthetic heart valve of FIG. 3 showing a leaflet in an open configuration.

FIG. 7 is a schematic, partial side view of the prosthetic heart valve of FIG. 3 showing a leaflet in a closed configuration.

FIG. 8 is a schematic, partial side view of the prosthetic heart valve of FIG. 3 showing two leaflets in a flattened arrangement for purposes of illustration.

### DETAILED DESCRIPTION

### General Considerations

For purposes of this description, certain aspects, advantages, and novel features of the examples of this disclosure are described herein. The described methods, systems, and apparatus should not be construed as limiting in any way. Instead, the present disclosure is directed toward all novel and non-obvious features and aspects of the various disclosed examples, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present, or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

### Examples of the Disclosed Technology

Described herein are examples of radially expandable and compressible prosthetic heart valves including an annular frame. In some examples, the frame of the prosthetic heart valve can include a plurality of rows of cells formed by interconnected struts of the frame. The plurality of rows of cells can be formed between an inflow end and an outflow end of the frame.

The prosthetic heart valve may further include a plurality of leaflets coupled to the frame. In some examples, the leaflets can include a fold pattern which defines inner layers and outer layers of leaflets.

In some examples, the outer layers of leaflets can remain relatively stationary with respect to the frame during the working cycle of the valve. The inner layers of leaflets can move inwardly and outwardly relative to the frame and contact the other inner layers of leaflets to regulate flow through the valve in one direction.

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

In some examples, the prosthetic heart valve can be radially expandable to a range of working diameters. For example, the prosthetic heart valve in the radially expanded state at a first working diameter can have outer layers of leaflets that are relatively longer than the inner layers of leaflets in a circumferential direction. When the prosthetic heart valve is expanded to a second larger working diameter, the inner layers of leaflets can lengthen, taking up slack from the outer layers of leaflets. At the second larger working diameter, the inner layers of leaflets are longer than the outer layers of leaflets in the circumferential direction. Thus, the inner layers of leaflets (e.g., the coaptation portions of the leaflets) can progressively increase in length in the circumferential direction as the prosthetic heart valve radially expands. This allows one prosthetic heart valve to accommodate a range of annulus sizes of patients.

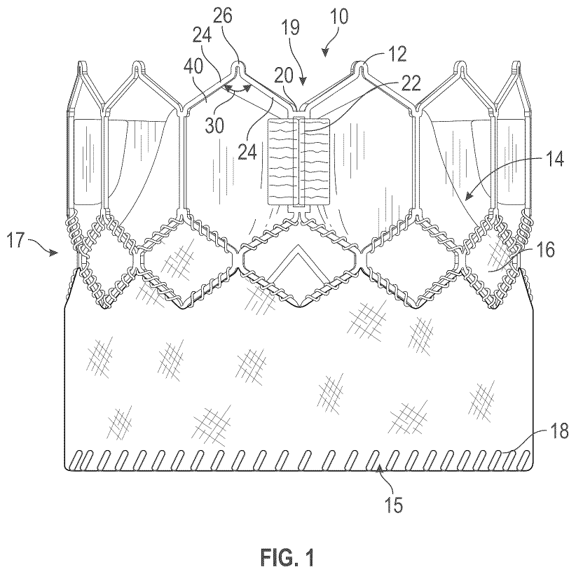

FIG. 1 shows an exemplary prosthetic valve 10, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 10 comprises four main components: a stent or frame 12, a valvular structure 14, an inner skirt 16, and a perivalvular outer sealing member or outer skirt 18. The prosthetic valve 10 can have an inflow end portion 15, an intermediate portion 17, and an outflow end portion 19. The inner skirt 16 can be arranged on and/or coupled to an inner surface of the frame 12, while the outer skirt 18 can be arranged on and/or coupled to an outer surface of the frame 12.

The valvular structure 14 can comprise three leaflets 40, collectively forming a leaflet structure, which can be arranged to collapse in a tricuspid arrangement, although in other examples there can be greater or fewer number of leaflets (e.g., one or more leaflets 40). The leaflets 40 can be secured to one another at their adjacent sides to form commissures 22 of the leaflet structure 14. The lower edge of valvular structure 14 can have an undulating, curved scalloped shape and can be secured to the inner skirt 16 by sutures (not shown). In some examples, the leaflets 40 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein. As described in more detail below, in some examples, the leaflets 40 can be folded to form neo-commissures that are circumferentially offset from the commissures 22. In these examples, the folding pattern of the leaflets 40 can enable the leaflets to be self-adjustable such that the leaflets 40 are operable to regulate blood flow at each diameter within a range of diameters.

The frame 12 can be radially compressible (collapsible) and expandable (e.g., expanded configuration shown in FIG. 1) and comprise a plurality of interconnected struts 24. A plurality of apices 26 that are spaced circumferentially apart are formed at the inflow end portion 15 and the outflow end portion 19 of the frame 12 (only the apices 26 at the outflow end portion 19 are visible in FIG. 1). Each apex 26 is formed at a junction between two angled struts 24 at either the inflow end portion 15 or the outflow end portion 19. FIG. 1 depicts a known frame design with apices 26 that form a U-shaped bend between the two angled struts 24. In some examples, an angle 30 between the two angled struts 24, connected at the apex 26, can be in a range of 90 to 120 degrees.

The frame 12 can be formed with a plurality of circumferentially spaced slots, or commissure windows 20 that are adapted to mount the commissures 22 of the valvular structure 14 to the frame. The frame 12 can be made of any of various suitable plastically expandable materials (e.g., stainless steel, etc.) or shape-memory, self-expanding materials (e.g., Nitinol). When constructed of a plastically expandable material, the frame 12 (and thus the prosthetic valve 10) can be crimped to a radially collapsed configuration on a delivery catheter or apparatus and then expanded inside a patient by an inflatable balloon or equivalent expansion mechanism. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frame 12 include metal alloys, polymers, or combinations thereof. Example metal alloys can comprise one or more of the following: nickel, cobalt, chromium, molybdenum, titanium, or other biocompatible metal. In some examples, the frame 12 can comprise stainless steel. In some examples, the frame 12 can comprise cobalt-chromium. In some examples, the frame 12 can comprise nickel-cobalt-chromium. In some examples, the frame 12 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight. Additional details regarding the prosthetic valve 10 and its various components are described in WIPO Patent Application Publication No. WO 2018/222799, which is incorporated herein by reference.

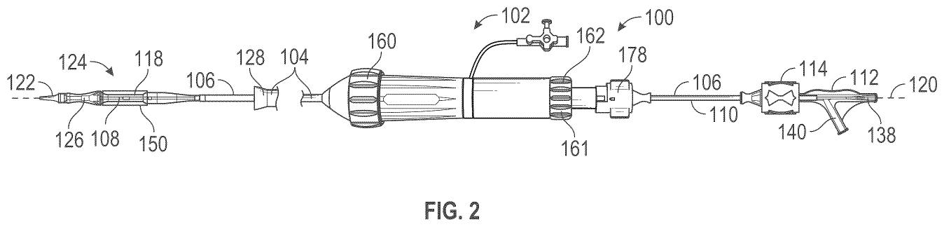

FIG. 2 shows a delivery apparatus 100, according to an example, that can be used to implant an expandable prosthetic heart valve (e.g., the prosthetic heart valve 10 of FIG. 1 and/or any of the other prosthetic heart valves described herein). In some examples, the delivery apparatus 100 is specifically adapted for use in introducing a prosthetic valve into a heart.

The delivery apparatus 100 in the illustrated example of FIG. 2 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (e.g., move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

The adaptor 112 can include a first port 138 configured to receive a guidewire therethrough and a second port 140 configured to receive fluid (e.g., inflation fluid) from a fluid source. The second port 140 can be fluidly coupled to an inner lumen of the intermediate shaft 106.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can be coupled to the distal end portion of the intermediate shaft 106.

In some examples, a distal end of the balloon 118 can be coupled to a distal end of the delivery apparatus 100, such as to a nose cone 122 (as shown in FIG. 2), or to an alternate component at the distal end of the delivery apparatus 100 (e.g., a distal shoulder). An intermediate portion of the balloon 118 can overlay a valve mounting portion 124 of a distal end portion of the delivery apparatus 100 and a distal end portion of the balloon 118 can overlay a distal shoulder 126 of the delivery apparatus 100. The valve mounting portion 124 and the intermediate portion of the balloon 118 can be configured to receive a prosthetic heart valve in a radially compressed state. For example, as shown schematically in FIG. 2, a prosthetic heart valve 150 (which can be one of the prosthetic valves described herein) can be mounted around the balloon 118, at the valve mounting portion 124 of the delivery apparatus 100.

The balloon shoulder assembly, including the distal shoulder 126, is configured to maintain the prosthetic heart valve 150 (or other medical device) at a fixed position on the balloon 118 during delivery through the patient's vasculature.

The outer shaft 104 can include a distal tip portion 128 mounted on its distal end. The outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 2) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 108 and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (e.g., for fine positioning at the implantation site). Further details on the delivery apparatus 100 can be found in PCT Application No. PCT/US2021/047056, which is incorporated by reference herein.

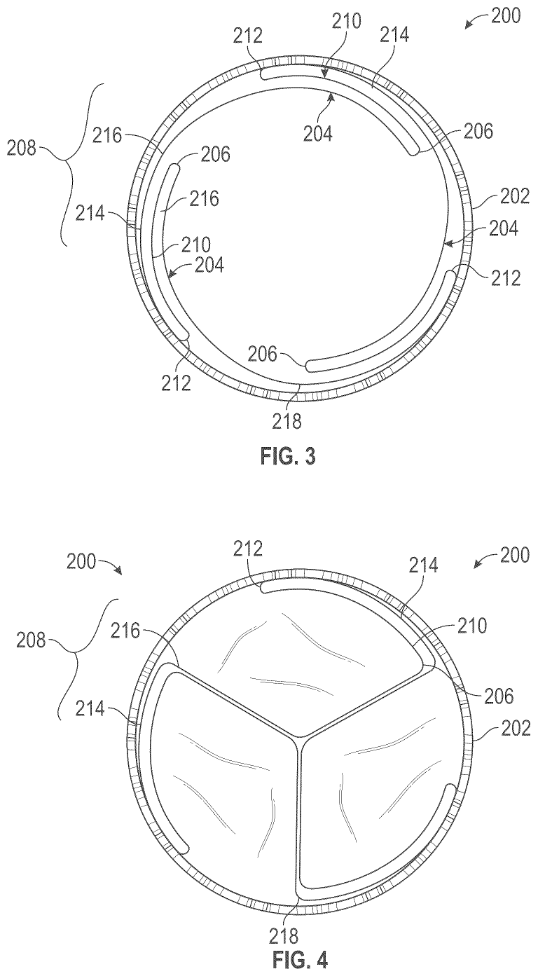

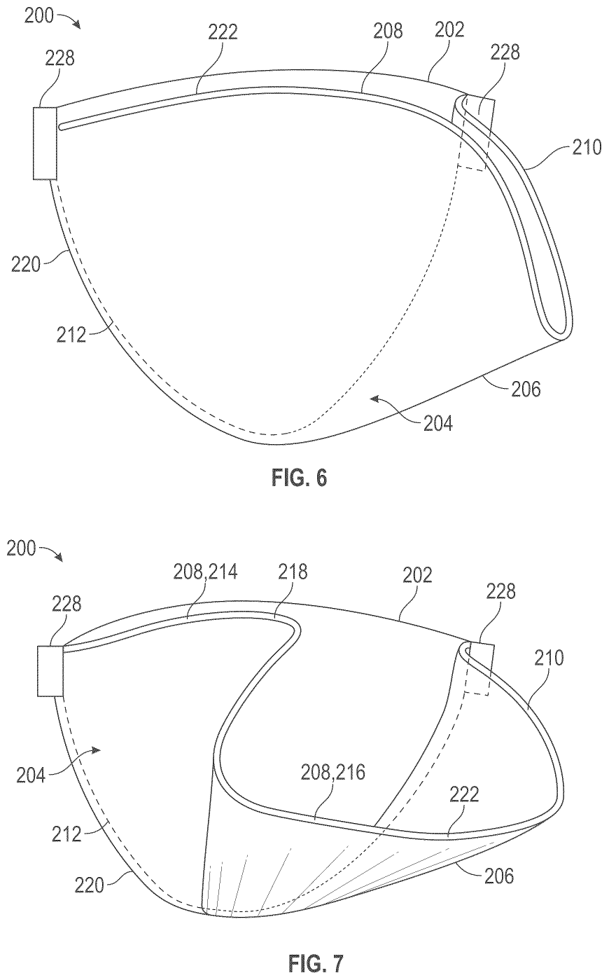

FIG. 3 shows an example of a prosthetic heart valve 200 comprising a radially expandable and compressible annular frame 202 and a valvular structure or leaflet assembly comprising a plurality of leaflets 204 (e.g., three in the illustrated example) coupled to the frame 202. Each leaflet 204 can comprise a longitudinal or axially extending fold 206. The fold 206 can extend axially from an inflow end toward an outflow end of the frame 202 (e.g., from an inflow or cusp edge portion of the leaflet 204 toward an outflow edge of the leaflet, etc.). In some examples, the fold 206 can extend parallel relative to a longitudinal axis of the valve 200. In some examples, the axially extending fold 206 can extend at an oblique angle relative to the longitudinal axis of the valve 200 (such as shown in FIG. 6). The axially extending fold 206 defines an inner layer 208 of the leaflet 204 and an outer layer 210 of the leaflet 204. At the fold 206, the inner layer 208 is disposed radially inward of the outer layer 210. Both the inner layer 208 and the outer layer 210 are positioned radially inward of the frame 202 (e.g., within the frame 202).

The frame 202 can be made of any of various suitable plastically expandable materials (e.g., stainless steel, cobalt-chromium alloy, etc.) or self-expanding materials (e.g., Nitinol). In some examples, the frame 202 comprises a plastically expandable material, such as any of those described above with reference to the prosthetic heart valve 10 of FIG. 1. In some examples, the frame 202 is similar to or the same as the frame 12 of FIG. 1.

The leaflets 204 can be directly or indirectly secured to the frame 202 with sutures. For example, the leaflets 204 can be secured to the frame 202 along a plurality of axially extending suture attachment lines 212 (also referred to as suture lines) defined by a plurality of stitches that secure axially extending side edges of the outer layers 210 of the leaflets directly to the frame 202. Along each suture attachment line 212, the stitches can be, for example, continuous in-and-out stitches that extend through an outer layer 210 of a leaflet 204 and through apertures (not shown) in the frame 202. Various other stitching techniques can be used. For example, the stitches can be whip stitches or loop stitches that extend through a leaflet and completely around a component of the frame 202 (for example, around selected struts of the frame). In other examples, the stitches in each suture attachment line 212 can be discrete (non-continuous) stitches. The suture attachment lines 212 desirably, although not necessarily, extend the entire height of the leaflets 204 from inflow edges of the leaflets 204 to outflow edges of the leaflets 204.

In other examples, the leaflets 204 can be coupled to the frame 202 via an inner skirt situated radially between the inner surface of the frame 202 and the outer surfaces of the leaflets 204. For example, the prosthetic valve 200 can include an inner skirt (e.g., a fabric skirt), similar to inner skirt 16, that is sutured to the frame 202 in the manner shown in FIG. 1 or otherwise attached to the frame 202. The leaflets 204 can be attached directly to the inner skirt along respective suture attachment lines 212 formed from stitches that extend through the leaflets 204 and the inner skirt, but need not extend around or through any components of the frame 202. In this manner, the cusp edge portion of each leaflet 204 is coupled to the frame via the inner skirt. In other examples, the cusp edge portions of the leaflets 204 can be connected directly to selected struts of the frame 202 (e.g., with sutures) and the inner skirt can be optional.

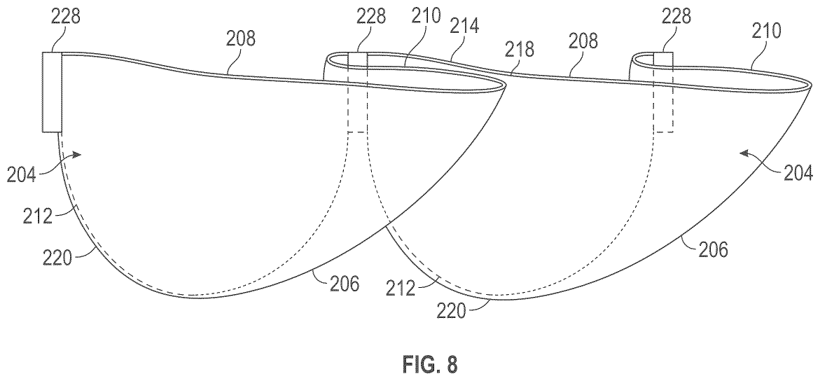

In some instances, the suture attachment line 212 can be u-shaped (scalloped shape) or v-shaped. For example, as shown in FIGS. 6-8, each leaflet 204 can have a cusp edge portion 220 (also referred to as an inflow edge portion) that generally corresponds to and tracks the shape of the suture line 212 from a first location at or adjacent the outflow edge 222 of the leaflet 204 to a second location at or adjacent the outflow edge 222 of the leaflet 204. FIGS. 6-7 only show one leaflet 204 of the prosthetic valve and FIG. 8 only shows two leaflets 204 of the prosthetic valve for purposes of illustration. The prosthetic valve in FIGS. 6-8 can include two or more leaflets 204 (such as three leaflets) folded in the same manner. The suture line 212 is illustrated in FIGS. 6-8 as a dashed line (e.g., indicating in-and-out stitches, etc.). In the views shown in FIGS. 6-8, portions of the suture line 212 are partially obstructed from view by the leaflets 204 and illustrated with a smaller dashed line.

In the example of FIGS. 6-8, each leaflet 204 can have commissure tabs 228, each of which is paired with an adjacent commissure tab 228 of an adjacent leaflet 204 to form a commissure that is connected to the frame 202, such as in the manner that commissures 22 (FIG. 1) are formed and connected to frame 12. The axially extending fold 206 can be formed at a location circumferentially between the commissure tabs 228 (and circumferentially between adjacent commissures). Each fold 206 extends from the cusp edge portion 220 to the outflow edge 222 of the leaflet. The cusp edge portion 220 of each leaflet 204 can extend from one commissure tab 228 to the other commissure tab 228. The cusp edge portion 220 can sutured to an inner skirt (such as inner skirt 16) along a suture line 212, with the inner skirt sutured or otherwise secured to the frame 202. Alternatively, the cusp edge portion 220 can be sutured directly to struts of the frame 202 along the suture line 212. In the views shown in FIGS. 6-8, one or more portions of the commissure tabs 238 are partially obstructed from view by the leaflets 204 and these portions are illustrated with a dashed line.

The leaflets 204 can be formed of one or more pieces of material. In the illustrated example, the leaflets 204 are formed from one, continuous piece of material (which can be referred to as a leaflet construct or valvular structure). As shown in FIG. 3, the piece of material forming the leaflets 204 is attached to the frame 202 with suture attachment lines 212 at three locations and folded in the manner shown in FIG. 3 to define three leaflets 204 between the sutures 212. Thus, each leaflet 204 is defined as a section of the leaflet construct between one suture attachment line 212 and a circumferentially adjacent suture attachment line 212. The leaflet construct can be rectangular and define rectangular leaflets between the suture attachment lines 212 or can define plural (e.g., three) v-shaped or u-shaped leaflets connected side-by-side. It should be appreciated that in other examples the prosthetic heart valve 200 may include a different number of leaflets (e.g., one or two leaflets 204 or greater than three leaflets).

In some examples, the leaflet construct or valvular structure can comprise a leaflet assembly comprising leaflets 204, each formed from a separate piece of material. In these examples, each longitudinal side edge of a leaflet 204 (an axially extending side edge) can be paired with an adjacent longitudinal side edge of an adjacent leaflet 204 and the pair of longitudinal side edges can be attached to the frame via a suture attachment line 212. Alternatively, adjacent longitudinal side edges of adjacent leaflets 204 can be circumferentially spaced apart from each other and attached to the frame 202 at separate, circumferentially spaced apart locations on the frame 202. For example, for a prosthetic valve having three leaflets 204, with each leaflet having two opposing longitudinal side edges, the frame 202 can have six leaflet attachment locations, with each longitudinal side edge being attached to a respective leaflet attachment location of the frame 202. In other examples, each separately formed leaflet 204 can be v-shaped or u-shaped (e.g., having v-shaped or u-shaped cusp edge portion) when in a flattened and fully unfolded state.

In some examples, each leaflet of a leaflet assembly can be formed from multiple pieces of material that are sutured or otherwise attached to each other. For example, the inner and outer layers 208, 210 of a leaflet 204 can be formed from separate piece of material.

In some examples, the leaflets 204 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

In some examples, each leaflet 204 can include more than one longitudinal fold 206 between the inner layer 208 and the outer layer 210 of a leaflet 204. For example, the leaflet 204 may be folded multiple times between the inner layer 208 and the outer layer 210 in an accordion fold pattern.

The inner layer 208 of each leaflet 204 can include a non-coaptation portion 214 and a coaptation portion 216. The non-coaptation portions 214 and the coaptation portions 216 of the leaflets 204 are also referred to herein as “non-coaptation segments” and “coaptation segments” of the leaflets 204, respectively. The transition between the non-coaptation portion 214 and the coaptation portion 216 of a leaflet 204 is aligned with a fold 206 of an adjacent leaflet 204. As described in more detail below, the non-coaptation portions 214 can remain relatively stationary relative to the frame 202 during a working cycle of the valve 200. The coaptation portions 216 can move inwardly and outwardly, in a radial direction, relative to the frame 202 during the working cycle of the valve 200 and coapt with the other coaptation portions 216 of the leaflets 204.

The non-coaptation portion 214 of the inner layer 208 of one leaflet 204 can be positioned radially outward of an outer layer 210 of an adjacent leaflet 204. Specifically, the non-coaptation portion 214 of one leaflet 204 and the outer layer 210 of an adjacent leaflet 204 are overlapped. The coaptation portion 214 of one leaflet 204 is positioned radially inward of an outer layer 210 of the same leaflet 204. Thus, stated differently, at certain locations, the leaflet construct can form three layers of leaflet material, namely, a non-coaptation portion 214 of a first leaflet 204, an outer layer 210 of a second leaflet 204 radially inward of the non-coaptation portion, and a coaptation potion 216 of the second leaflet 204 radially inwardly of the outer layer. The outer layers 210 and the non-coaptation portions 214 of the leaflets 204 provide additional material or slack that can be used by the coaptation portions 216 of the leaflets 204 to increase the length of the coaptation portions 216 when the prosthetic heart valve 200 is radially expanded, as described in more detail below.

As shown in FIG. 3, the folds 206 are circumferentially offset from the suture attachment lines 212. A location 218 where the transition between the non-coaptation portion 214 and the coaptation portion 216 of the inner layer 208 of a first leaflet 204 is aligned with a fold 206 of a second leaflet defines a commissure for the first and second leaflets. This commissure 218 can be referred to as a “neo-commissure” of the first and second leaflets because its location along the inner layer and the spacing between adjacent commissure can vary depending on the extent that the frame is radially expanded, as further described below. The coaptation portion 216 of each leaflet can articulate at a corresponding fold 206 defining a first neo-commissure 218 and at a second neo-commissure 218. In this example, the neo-commissures 218 are circumferentially offset from the location where ends of two adjacent leaflets 204 meet (e.g., at sutures 212).

During working cycles of the valve 200, the non-coaptation portion 214 of the inner layer 208 of each leaflet 204 remains substantially stationary relative to the frame 202. The outer layer 210 of each leaflet 204 also remains substantially stationary relative to the frame 202. The coaptation portion 216 of the inner layer 208 can move inwardly and outwardly relative to the outer layer 210 at the neo-commissures 218. The coaptation portion 216 of the inner layer 208 defines the coaptation edge of the leaflet 204 that can contact the coaptation edges of the other leaflets 204.

Overtime, tissue ingrowth can occur along the non-coaptation portions 214 to secure the non-coaptation portions 214 to the inner surface of the frame 202. In some examples, the tissue ingrowth can propagate through the non-coaptation portions 214 and occur along the outer layers 210 to secure the outer layers 210 to the non-coaptation portions 214 and to the inner surface of the frame 202. In some examples, an anchor (e.g., a staple, a suture, etc.) can be used to secure the non-coaptation portions 214 and/or the outer layers 210 to the inner surface of the frame 202 after the prosthetic valve is implanted within a patient.

FIGS. 3-4 illustrate two stages of a working cycle of the prosthetic heart valve 200. Specifically, FIG. 3 shows a first stage of the working cycle with the leaflets 204 of the prosthetic heart valve 200 in an open configuration, and FIG. 4 shows a second stage of the working cycle with the leaflets 204 of the prosthetic heart valve 200 in a closed configuration. FIGS. 6-7 illustrate side views of the two stages of the working cycle of the prosthetic heart valve 200. Specifically, FIG. 6 shows the first stage of the working cycle when the leaflets 204 of the prosthetic heart valve 200 are in an open configuration, and FIG. 7 shows a second stage of the working cycle when the leaflets 204 of the prosthetic heart valve 200 are in a closed configuration (although FIGS. 6 and 7 show only one leaflet 204, the other leaflets 204 would move in the same manner between the open and closed configurations). As shown in FIGS. 3-4 and 6-7, the outer layers 210 and the non-coaptation portions 214 of the leaflets 204 are in substantially the same location in the open configuration and in the closed configuration. In some examples, this is due to tissue ingrowth over the outer layers 210 and the non-coaptation portions 214 of the leaflets 204. In the closed configuration, the coaptation portions 216 of the leaflets 204 are in a position that is radially inward relative to the frame 202, such that the coaptation portion 216 of one leaflet 204 contacts the coaptation portions 216 of the other leaflets 204. With the leaflets 204 in the closed configuration, blood is prevented from passing through the valve 200. During the working cycle, the prosthetic heart valve 200 alternates between the open configuration and the closed configuration. This enables the prosthetic heart valve 200 to regulate the flow of blood in one direction.

To accommodate a range of working diameters, the leaflets 204 are self-adjustable as the frame 202 expands. When the frame 202 is in a radially compressed state, the outer layer 210 of each leaflet 204 can be relatively longer in the circumferential direction than the inner layer 208 along the outflow edge 222. As the frame 202 is radially expanded during the implantation procedure, the length of the inner layer 208 in the circumferential direction increases along the outflow edge 222 while the length of the outer layer 210 shortens along the outflow edge 222 due to the presence of the fold 206. Stated differently, the outer layer 210 provides slack that is taken up by the inner layer 208 along the outflow edge 222 as the frame 202 radially expands. As described above, the cusp edge or inflow edge portion 220 of each leaflet 204 is secured to the frame 202 at the suture attachment line 212. As a result, as the frame radially expands, the curvature of the inflow edge portions 220 decreases and the length of the inflow edge portions 220 of the leaflets 204 do not change. Moreover, as the frame 202 is radially expanded, the locations and spacing between neo-commissures 218 varies due to the presence of the folds 206.

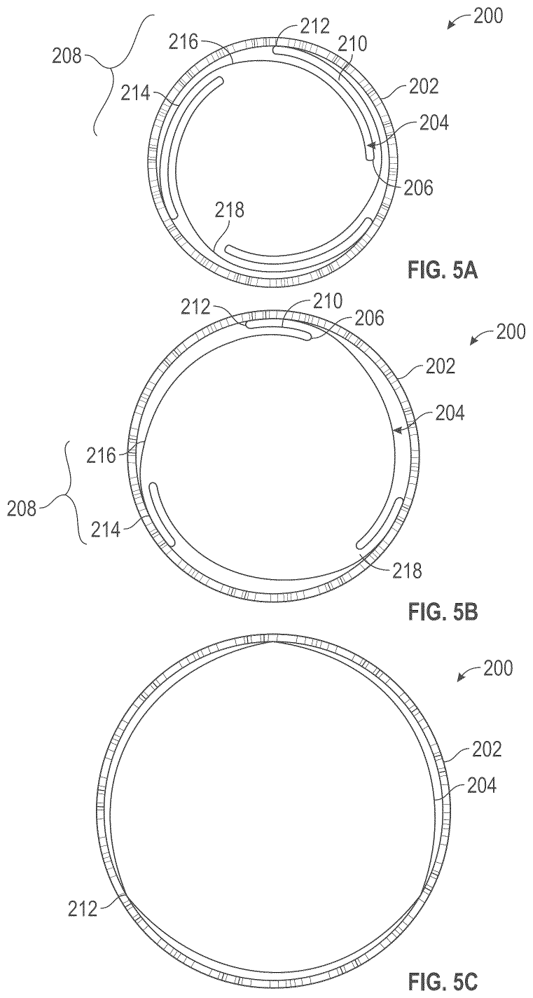

FIGS. 5A-5C show an example of the self-adjustment of the leaflets 204 as the frame 202 of the prosthetic heart valve 200 radially expands from a first working diameter (FIG. 5A), to a second, larger working diameter (FIG. 5B), and to a third, maximum working diameter (FIG. 5C). FIGS. 5A-5C show the leaflets 204 in an open configuration. Due to the nature of the folds 206, the prosthetic heart valve 200 includes the same number of leaflets 204 at each diameter within the range of working diameters (e.g., in a compressed state, in an expanded state, at each diameter, etc.). In the illustrated examples, three leaflets 204 are shown.

As introduced above, the prosthetic valve 200 can be expanded to a working diameter within a range of working diameters (e.g., 20 mm to 30 mm, 25 mm to 40 mm, 40 mm to 60 mm, etc.). At each working diameter within the range, the leaflets 204 are configured to regulate the flow of blood through the valve 200 in one direction. Specifically, the coaptation portions 216 of the inner layers 208 of the leaflets 204 move inwardly and outwardly in a radial direction relative to the outer layers 210 at the folds 206 and the coaptation portions 216 contact each other, thereby preventing the flow of blood through the valve 200 in one direction.

FIG. 5A shows the prosthetic heart valve 200 expanded to a first working diameter (e.g., a diameter at a lower end of the range of working diameters, etc.). In this example, a significant portion of the leaflets 204 are overlapped and the fold 206 is offset in a circumferential direction from a corresponding suture attachment line 212. As shown, the coaptation portion 216 of each leaflet 204 is relatively shorter in length than the rest of the leaflet 204 (e.g., the non-coaptation portion 214 and the outer layer 210).

FIG. 5B shows the prosthetic heart valve 200 expanded to a second, larger working diameter. Rather than having incremental steps between diameters, the prosthetic heart valve 200 can be progressively increased to a desired diameter within a range of diameters during initial implantation of the prosthetic valve 200. In this way, the prosthetic heart valve 200 smoothly transitions from one working diameter to the next. Specifically, as the valve 200 is expanded (e.g., by inflating balloon 118, actuating one or more actuators of the delivery apparatus, or deploying the prosthetic valve from a sheath to allow the prosthetic valve to self-expand), the inner layers 208 of the leaflets 204 slide relative to the outer layers 210 of the leaflets 204, which moves the fold 206 in a circumferential direction towards the suture attachment line 212. Similarly, the neo-commissures 218 also move in a circumferential direction towards the suture attachment line 212. As shown in the illustrated example, a distance between adjacent folds 206 is shorter in FIG. 5A when the frame 202 is expanded to the first diameter than the distance between the adjacent folds 206 in FIG. 5B when the frame 202 is expanded to the second diameter. Similarly, a distance between adjacent neo-commissures 218 is shorter in FIG. 5A when the frame 202 is expanded to the first diameter than the distance between the adjacent neo-commissures 218 in FIG. 5B when the frame 202 is expanded to the second diameter.

Radially expanding the prosthetic heart valve 200 causes the offset between the suture attachment line 212 and the fold 206 to decrease, resulting in a smaller portion of the leaflets 204 being overlapped. In other words, as the frame 202 radially expands, the coaptation portions 216 of the leaflets 204 increase in length and the overlapped outer layer 210 and non-coaptation portions 214 decrease in length. During this expansion, the coaptation portion 216 uses or takes up material from the outer layer 210 and the non-coaptation portion 214 of the leaflet 204 to achieve this increased length.

As shown in FIG. 5C, at the upper end of the range, the leaflets 204 can completely unfold such that each leaflet 204 forms a single layer. At the maximum working diameter within the range, the entire length of the leaflets 204 coapt with the other leaflets 204. In this example, the leaflets 204 no longer include inner and outer layers, and no longer include folds. Consequently, the commissures of the leaflets are located at the suture attachment lines 212.

Thus, a single prosthetic valve 200 can be implanted in patients having a range of annulus sizes. With known prosthetic heart valves, a hospital typically stocks between three and four different sizes of the same type of valve (e.g., a 20-mm valve, a 23-mm valve, a 26-mm valve, and a 29-mm valve) to treat patients with different annulus sizes. Advantageously, a single valve 200 can be used to treat the patients across a range of patient annulus sizes (e.g., 20 mm to 30 mm). Thus, a hospital can stock a single type of valve 200 to treat the same number of patients instead of three or four different sizes of the same valve.

Moreover, in some implementations, the prosthetic valve 200 can be configured to be further expanded and/or can further self-expand at some time after it is initially implanted to accommodate growth of the patient, for example, days, weeks, months, or years after the initial implantation. For example, if the frame 202 is formed from a plastically-expandable material, the prosthetic valve can be further expanded in a subsequent procedure after its initial implantation by delivering a catheter having an expansion mechanism (e.g., an inflatable balloon) into the body and positioning the expansion mechanism within the prosthetic valve. The expansion mechanism can then be expanded to further expand the prosthetic valve to a larger diameter (causing the leaflets to automatically adjust and create longer leaflet coaptation portions) that permits a greater amount of blood flow through the prosthetic valve. If the frame 202 is formed from a shape-memory (self-expandable) material (e.g., Nitinol), the frame can self-expand over time as the size of the native annulus increases. As the frame self-expands over time, the leaflets automatically adjust (the coaptation portions become longer) to the larger size of the frame. In some cases, even with a self-expandable frame, it may be necessary or desirable to use a catheter with an expansion mechanism in a subsequent procedure to further expand the prosthetic valve or to assist the radial expansion of the frame.

In some such examples, the prosthetic valve can include an outer skirt (e.g., outer skirt 18) extending around the outer surface of the frame to prevent or minimize tissue ingrowth on the non-coaptation portions 214 and/or the outer layers 210 of the leaflets to allow the leaflet to self-adjust as the prosthetic valve is further expanded.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded to a diameter within a range of working diameters (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Simulation

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: a radially expandable frame comprising a plurality of interconnected struts; and a plurality of leaflets disposed within the frame and configured to regulate a flow of blood through the frame in one direction, wherein each leaflet is folded along an axially extending fold to form an inner layer and an outer layer, wherein the inner layer of each leaflet is configured to coapt with the inner layers of the other leaflets, and wherein a length of the inner layer in a circumferential direction progressively increases upon radial expansion of the frame.

Example 2. The prosthetic heart valve of any example herein, particularly example 1, wherein a length of the outer layer in a circumferential direction progressively decreases upon radial expansion of the frame.

Example 3. The prosthetic heart valve of any example herein, particularly either example 1 or example 2, wherein the leaflets have cusp edge portions that are coupled to the frame with sutures.

Example 4. The prosthetic heart valve of any example herein, particularly example 3, wherein cusp edge portions are sutured to an inner skirt disposed between the leaflets and an inner surface of the frame.

Example 5. The prosthetic heart valve of any example herein, particularly any one of examples 1-4, wherein the cusp edge portions are generally v-shaped or u-shaped.

Example 6. The prosthetic heart valve of any example herein, particularly any one of examples 1-5, wherein each leaflet is formed from a separate piece of material.

Example 7. The prosthetic heart valve of any example herein, particularly any one of examples 1-5, wherein the plurality of leaflets is formed from one continuous piece of material.

Example 8. The prosthetic heart valve of any example herein, particularly any one of examples 1-7, wherein the plurality of leaflets is operable to regulate the flow of blood through the frame in one direction at each diameter of a range of working diameters of the frame.

Example 9. The prosthetic heart valve of any example herein, particularly example 8, wherein each leaflet is unfolded at a maximum working diameter of the range of working diameters.

Example 10. A prosthetic heart valve comprising: a frame comprising a plurality of interconnected struts, wherein the frame is radially expandable between at least a first diameter and a larger, second diameter; and a plurality of leaflets disposed within the frame and configured to regulate a flow of blood through the frame in one direction when the frame is radially expanded to the first diameter and when the frame is radially expanded to the second diameter, wherein each leaflet is folded along a longitudinal fold to form an inner layer and an outer layer, wherein each longitudinal fold defines a commissure for a pair of adjacent inner layers of two adjacent leaflets, wherein the inner layer of each leaflet defines a coaptation edge configured to move inwardly and outwardly in a radial direction relative to the outer layer at the commissure.

Example 11. The prosthetic heart valve of any example herein, particularly example 10, wherein a length of the inner layer of each leaflet in a circumferential direction increases when the frame is radially expanded from the first diameter to the second diameter.

Example 12. The prosthetic heart valve of any example herein, particularly either example 10 or example 11, wherein a length of the outer layer of each leaflet in a circumferential direction decreases when the frame is radially expanded from the first diameter to the second diameter.

Example 13. The prosthetic heart valve of any example herein, particularly example 12, wherein the inner layer of each leaflet takes up material of the outer layer at the fold such that the length of the inner layer increases and the length of the outer layer decreases as the frame is radially expanded.

Example 14. The prosthetic heart valve of any example herein, particularly any one of examples 10-13, wherein a distance between adjacent folds in a circumferential direction increases as the frame is radially expanded.

Example 15. The prosthetic heart valve of any example herein, particularly any one of examples 10-14, wherein a distance between adjacent commissures in a circumferential direction increases as the frame is radially expanded.

Example 16. The prosthetic heart valve of any example herein, particularly any one of examples 10-15, wherein a fold of one of the plurality of leaflets is positioned radially inward of a portion of an adjacent leaflet.

Example 17. The prosthetic heart valve of any example herein, particularly any one of examples 10-16, wherein the prosthetic heart valve includes the same number of leaflets when the frame is radially expanded to the first diameter and when the frame is radially expanded to the second diameter.

Example 18. The prosthetic heart valve of any example herein, particularly any one of examples 10-17, wherein the frame is radially expandable to a third diameter that is larger than the second diameter; and wherein the plurality of leaflets is configured to regulate the flow of blood through the frame in one direction when the frame is radially expanded to the third diameter.

Example 19. The prosthetic heart valve of any example herein, particularly any one of examples 10-18, wherein the plurality of leaflets is formed from one continuous piece of material.

Example 20. A prosthetic heart valve, comprising: a radially expandable frame comprising a plurality of interconnected struts; and a plurality of self-adjustable leaflets disposed within the frame, each leaflet comprising a longitudinal fold defining an outer layer and an inner layer, wherein an inner layer of a first leaflet includes a non-coaptation portion and a coaptation portion, wherein the non-coaptation portion of the first leaflet is disposed radially outward of an outer layer of a second leaflet, wherein each coaptation portion is configured to move inwardly and outwardly in a radial direction relative to the outer layer at the longitudinal fold.

Example 21. The prosthetic heart valve of any example herein, particularly example 20, wherein the longitudinal fold of the first leaflet is aligned with an end of the coaptation portion of the second leaflet to define a commissure between the first leaflet and the second leaflet.

Example 22. The prosthetic heart valve of any example herein, particularly example 21, wherein a distance between adjacent commissures in a circumferential direction increases as the frame is radially expanded.

Example 23. The prosthetic heart valve of any example herein, particularly any one of examples 20-22, wherein the non-coaptation portion of the first leaflet is connected to the outer layer of the second leaflet.

Example 24. The prosthetic heart valve of any example herein, particularly any one of examples 20-23, wherein a length of the inner layer linearly increases as the frame radially expands and a length of the outer layer linearly decreases as the frame radially expands.

Example 25. The prosthetic heart valve of any example herein, particularly any one of examples 20-24, wherein the plurality of leaflets is formed from one continuous piece of material.

Example 26. The prosthetic heart valve of any example herein, particularly any one of examples 20-25, wherein an outer layer of the first leaflet is disposed radially outward of the inner layer of the first leaflet at the longitudinal fold.

Example 27. A prosthetic heart valve, comprising: a frame including a plurality of leaflet attachment members; and a leaflet construct disposed within the frame and attached to the frame at the plurality of leaflet attachment members, the leaflet construct defining a leaflet between each of the plurality of leaflet attachment members, wherein each leaflet includes a longitudinal fold defining an inner layer and an outer layer, wherein each fold is circumferentially offset from the plurality of leaflet attachment members, wherein the inner layers of the leaflets are configured to move inward and outward in a radial direction relative to the frame to regulate the flow of blood through the frame.

Example 28. The prosthetic heart valve of any example herein, particularly example 27, wherein the leaflet construct is attached to the plurality of leaflet attachment members with sutures.

Example 29. The prosthetic heart valve of any example herein, particularly either example 27 or example 28, wherein the frame is radially expandable between a range of diameters; and wherein a circumferential length of the offset between the fold and one of the leaflet attachment members decreases as the frame radially expands from a first diameter within the range of diameters to a second, larger diameter within the range of diameters.

Example 30. The prosthetic heart valve of any example herein, particularly example 29, wherein a circumferential length of the inner layer increases as the frame radially expands from the first diameter to the second diameter.

Example 31. The prosthetic heart valve of any example herein, particularly either example 29 or example 30, wherein a circumferential length of the outer layer decreases as the frame radially expands from the first diameter to the second diameter.

Example 32. The prosthetic heart valve of any example herein, particularly any of examples 27-31, wherein the leaflet construct comprises a single, continuous piece of material define all of the leaflets.

Example 33. The prosthetic heart valve of any example herein, particularly any of examples 27-31, wherein the leaflet construct comprises separate pieces of material, each forming one of the leaflets.

Example 34. A prosthetic heart valve, comprising: a radially expandable frame; and a leaflet construct disposed within the frame and including a plurality of leaflets, each leaflet including a longitudinal fold defining an inner layer and an outer layer, each inner layer having a non-coaptation segment and a coaptation segment, wherein a first outer layer is connected to a coaptation segment of a first inner layer and a non-coaptation segment of a second inner layer, wherein the first outer layer is disposed radially outward of the coaptation segment of the first inner layer, wherein the first outer layer is disposed radially inward of the non-coaptation segment of the second inner layer, wherein a length of the inner layers in a circumferential direction progressively increases upon radial expansion of the leaflet assembly, wherein a length of the outer layers in a circumferential direction progressively decreases upon radial expansion of the leaflet assembly.

Example 35. The prosthetic heart valve of any example herein, particularly example 34, wherein a second outer layer is connected to a coaptation segment of the second inner layer and a non-coaptation segment of a third inner layer, wherein the second outer layer is disposed radially inward of the non-coaptation segment of the third inner layer.

Example 36. The prosthetic heart valve of any example herein, particularly either example 34 or example 35, wherein the frame is expandable within a range of working diameters.

Example 37. The prosthetic heart valve of any example herein, particularly any one of examples 34-36, wherein the first inner layer takes up material of the first outer layer at the fold such that a length of the first inner layer increases and a length of the first outer layer decreases as the frame is radially expanded.

Example 38. The prosthetic heart valve of any example herein, particularly any one of examples 34-37, further comprising sutures connecting the plurality of leaflets to the frame.

Example 39. The prosthetic heart valve of any example herein, particularly any one of examples 34-38, wherein the plurality of leaflets is formed from one continuous piece of material.

Example 40. A leaflet construct for a prosthetic heart valve, comprising: three inner layers, each inner layer having a non-coaptation segment and a coaptation segment; three outer layers coupled to the three inner layers, wherein a first outer layer of the three outer layers is connected to a coaptation segment of a first inner layer and a non-coaptation segment of a second inner layer, wherein the first outer layer is disposed radially outward of the coaptation segment of the first inner layer, wherein a second outer layer of the three outer layers is connected to a coaptation segment of the second inner layer and a non-coaptation segment of a third inner layer, wherein the second outer layer is disposed radially inward of the non-coaptation segment of the third inner layer; at least one longitudinal fold positioned between each inner layer and a corresponding outer layer; wherein a length of the inner layers in a circumferential direction progressively increases upon radial expansion of the leaflet construct; and wherein a length of the outer layers in a circumferential direction progressively decreases upon radial expansion of the leaflet construct.

Example 41. The leaflet construct of any example herein, particularly example 40, wherein the coaptation segment of the second inner layer is configured to move inwardly and outwardly in a radial direction relative to the second outer layer under the flow of blood.

Example 42. The leaflet construct of any example herein, particularly either example 40 or example 41, wherein the second inner layer takes up material of the second outer layer at the fold such that a length of the second inner layer increases and a length of the second outer layer decreases as the leaflet construct is radially expanded.

Example 43. A delivery assembly comprising: a delivery apparatus including an expansion mechanism; and a prosthetic heart valve coupled to the delivery apparatus, the prosthetic heart valve comprising: a radially expandable frame; and a plurality of leaflets disposed within the frame and configured to regulate a flow of blood through the frame in one direction, wherein each leaflet is folded along a longitudinal fold to form an inner layer and an outer layer, wherein the inner layer of each leaflet is configured to coapt with the inner layers of the other leaflets, and wherein a length of the inner layer in a circumferential direction progressively increases upon radial expansion of the frame by the expansion mechanism.

Example 44. The delivery assembly of any example herein, particularly example 43, wherein the expansion mechanism is one of: a balloon on which the prosthetic heart valve is mounted, a mechanical actuator that applies an expansion force to the prosthetic heart valve, and a sheath of the delivery apparatus which deploys the prosthetic heart valve so that the prosthetic heart valve self-expands to its functional size.

Example 45. The delivery assembly of any example herein, particularly either example 43 or example 44, wherein a distance between adjacent folds in a circumferential direction increases as the frame is radially expanded.

Example 46. The delivery assembly of any example herein, particularly any one of examples 43-45, wherein the frame is radially expandable to each diameter of a range of diameters; and wherein the plurality of leaflets is configured to regulate the flow of blood through the frame when the frame is expanded to each diameter of the range of diameters.

Example 47. The delivery assembly of any example herein, particularly example 46, wherein the range of diameters is 20 mm to 30 mm.

Example 48. The delivery assembly of any example herein, particularly example 46, wherein the range of diameters is 25 mm to 40 mm.

Example 49. The delivery assembly of any example herein, particularly example 46, wherein the range of diameters is 40 mm to 60 mm.

Example 50. A method of implantation, comprising: inserting a delivery device into a vessel of a patient, the delivery device including a radially expandable prosthetic heart valve; and expanding the prosthetic heart valve within or adjacent a native valve of a heart of the patient to a first diameter of a range of diameters of the prosthetic heart valve, the prosthetic heart valve including leaflets having folds defining inner layers of leaflets and outer layers of leaflets which slide relative to each other upon expansion of the prosthetic heart valve.

Example 51. The method any example herein, particularly of example 50, wherein expanding the prosthetic heart valve to the first diameter comprises increasing a length of the inner layers of leaflets in a circumferential direction and decreasing a length of the outer layers of leaflets in a circumferential direction.

Example 52. The method any example herein, particularly of either example 50 or example 51, further comprising expanding the prosthetic heart valve to a second diameter of the range of diameters, wherein the second diameter is larger than the first diameter.

Example 53. The method any example herein, particularly of example 52, wherein expanding the prosthetic heart valve to the second diameter comprises unfolding the folds of the leaflets.

Example 54. A method of delivering an implant within a patient's body, the implant comprising a prosthetic heart valve of any of examples 1-35, the method comprising: positioning the prosthetic heart valve at an implantation site within the patient's body; expanding the prosthetic heart valve to a working diameter within a range of working diameters of the prosthetic heart valve.

Example 55. The method of any example herein, particularly example 54, wherein expanding the prosthetic heart valve to the working diameter comprises sliding an inner layer of a leaflet relative to an outer layer of the leaflet at a fold of the leaflet, such that the inner layer increases in length and the outer layer decreases in length.

Example 56. The method of any example herein, particularly either example 54 or example 55, further comprising expanding the prosthetic heart valve to a larger working diameter.

Example 57. The method of any example herein, particularly example 56, further comprising unfolding the leaflets.

Example 58. A prosthetic heart valve of any example herein, particularly, any of examples 1-39, wherein the prosthetic heart valve is sterilized.

Example 59. A leaflet construct of any example herein, particularly, any of examples 40-42, wherein the leaflet construct is sterilized.

Example 60. A delivery assembly of any example herein, particularly, any of examples 43-49, wherein the delivery assembly is sterilized.

The features described herein with regard to any example can be combined with other features described in any one or more of the other examples, unless otherwise stated. For example, any one or more of the features of one prosthetic heart valve can be combined with any one or more features of another prosthetic heart valve.

In view of the many possible examples to which the principles of the disclosed technology may be applied, it should be recognized that the illustrated examples are only preferred examples of the disclosed technology and should not be taken as limiting the scope of the claimed subject matter. Rather, the scope of the claimed subject matter is defined by the following claims and their equivalents.

1. A prosthetic heart valve comprising:
a radially expandable frame comprising a plurality of interconnected struts; and
a plurality of leaflets disposed within the frame and configured to regulate a flow of blood through the frame in one direction, wherein each leaflet is folded along an axially extending fold to form an inner layer and an outer layer, wherein the inner layer of each leaflet is configured to coapt with the inner layers of the other leaflets,
wherein a length of the inner layer in a circumferential direction progressively increases upon radial expansion of the frame, and
wherein a length of the outer layer in a circumferential direction progressively decreases upon radial expansion of the frame.

2. The prosthetic heart valve of claim 1, wherein the leaflets have cusp edge portions that are coupled to the frame with sutures.

3. The prosthetic heart valve of claim 2, wherein cusp edge portions are sutured to an inner skirt disposed between the leaflets and an inner surface of the frame.

4. The prosthetic heart valve of claim 2, wherein the cusp edge portions are generally v-shaped or u-shaped.

5. The prosthetic heart valve of claim 1, wherein each leaflet is formed from a separate piece of material.

6. The prosthetic heart valve of claim 1, wherein the plurality of leaflets is formed from one continuous piece of material.

7. The prosthetic heart valve of claim 1, wherein the plurality of leaflets is operable to regulate the flow of blood through the frame in one direction at each diameter of a range of working diameters of the frame.

8. The prosthetic heart valve of claim 7, wherein each leaflet is unfolded at a maximum working diameter of the range of working diameters.

9. The prosthetic heart valve of claim 1 wherein the plurality of leaflets comprises three leaflets.

10. A leaflet construct for a prosthetic heart valve comprising:
three inner layers, each inner layer having a non-coaptation segment and a coaptation segment;
three outer layers coupled to the three inner layers, wherein a first outer layer of the three outer layers is connected to a coaptation segment of a first inner layer and a non-coaptation segment of a second inner layer, wherein the first outer layer is disposed radially outward of the coaptation segment of the first inner layer, wherein a second outer layer of the three outer layers is connected to a coaptation segment of the second inner layer and a non-coaptation segment of a third inner layer, wherein the second outer layer is disposed radially inward of the non-coaptation segment of the third inner layer;
at least one longitudinal fold positioned between each inner layer and a corresponding outer layer;
wherein a length of the inner layers in a circumferential direction progressively increases upon radial expansion of the leaflet construct; and
wherein a length of the outer layers in a circumferential direction progressively decreases upon radial expansion of the leaflet construct.

11. The leaflet construct of claim 10, wherein the coaptation segment of the second inner layer is configured to move inwardly and outwardly in a radial direction relative to the second outer layer under the flow of blood.

12. The leaflet construct of claim 10, wherein the second inner layer takes up material of the second outer layer at the fold such that a length of the second inner layer increases and a length of the second outer layer decreases as the leaflet construct is radially expanded.

13. A delivery assembly comprising:
a delivery apparatus including an expansion mechanism; and
a prosthetic heart valve coupled to the delivery apparatus, the prosthetic heart valve comprising:
a radially expandable frame; and
a plurality of leaflets disposed within the frame and configured to regulate a flow of blood through the frame in one direction, wherein each leaflet is folded along a longitudinal fold to form an inner layer and an outer layer, wherein the inner layer of each leaflet is configured to coapt with the inner layers of the other leaflets, and wherein a length of the inner layer in a circumferential direction progressively increases upon radial expansion of the frame by the expansion mechanism.

14. The delivery assembly of claim 13, wherein the expansion mechanism is one of: a balloon on which the prosthetic heart valve is mounted, a mechanical actuator that applies an expansion force to the prosthetic heart valve, and a sheath of the delivery apparatus which deploys the prosthetic heart valve so that the prosthetic heart valve self-expands to its functional size.

15. The delivery assembly of claim 13, wherein a distance between adjacent folds in a circumferential direction increases as the frame is radially expanded.

16. The delivery assembly of claim 13, wherein the frame is radially expandable to each diameter of a range of diameters; and
wherein the plurality of leaflets is configured to regulate the flow of blood through the frame when the frame is expanded to each diameter of the range of diameters.

17. The delivery assembly of claim 16, wherein the range of diameters is 20 mm to 30 mm.

18. The delivery assembly of claim 16, wherein the range of diameters is 25 mm to 40 mm.

19. The delivery assembly of claim 16, wherein the range of diameters is 40 mm to 60 mm.
